(19) United States
(12) Patent Application Publication
(10) Pub. No.: US 2022/0202750 A1
(43) Pub. Date: Jun. 30, 2022

(54) DYE COMPOSITION FOR MARKING ORGANIC TISSUE WITH ANAESTHETIC AND METHOD FOR APPLYING SAME

(71) Applicants: Juliana SANDIN, São José (BR); Daniel VOLPATO, Florianópolis (BR); Rodrigo SANDIN, São José (BR); Doris Maria HEXSEL, Porto Alegre (BR)

(72) Inventors: Juliana SANDIN, São José (BR); Daniel VOLPATO, Florianópolis (BR); Rodrigo SANDIN, São José (BR)

(21) Appl. No.: 17/603,465

(22) PCT Filed: Apr. 18, 2020
(86) PCT No.: PCT/BR2020/050133

§ 371 Date: Oct. 13, 2021

(30) Foreign Application Priority Data
Apr. 18, 2019 (BR) 10 2019 007986 0
Apr. 18, 2020 (BR) 10 2020 007792 9

(51) Int. Cl. A61K 31/167; A61K 31/245; A61K 45/06; A61K 47/02; A61P 23/02

(52) U.S. Cl. CPC A61K 31/167; A61K 31/245; A61P 23/02; A61K 47/02; A61K 45/06

(57) ABSTRACT

The present invention relates to the technical field of dyes for marking organic tissue. It relates to a dye whose composition contains pigments, topical anaesthetics and absorption potentiator, used to visually mark a specific area of the surface of the organic tissue and concomitantly anaesthetize the marked area. The anaesthetic dye can be applied with a pen/stick. The invention simplifies procedures that require the marking of an anaesthetized region for subsequent intervention, saving time and anaesthetic and providing greater safety for patients. A method for applying said composition is also provided.

### FIELD OF THE INVENTION

In a general manner, the present invention belongs to the technological sector of the pharmaceutical industry and refers, more specifically, to a dye for marking organic tissue that has in its composition, besides pigments, topical anesthetics for use on live beings. With this composition, said dye serves to mark a specific area of the organic tissue and concomitantly anesthetize the marked area. The present invention further provides a method for applying this composition.

### BRIEF DESCRIPTION OF THE INVENTION

In invasive or minimally invasive medical, surgical and cosmetic procedures on biological tissue, such as those involving puncture, scarification, exeresis, or any other procedure that causes pain or discomfort, the use of topical anesthetics is commonplace, besides marking the region of the tissue with the aim of precisely specifying the point or area where the procedure will occur. Today, and routinely, these procedures are carried out using anesthetic ointments or creams throughout the area where the procedure will be carried out, which means that a large amount of the product is used over a large area of organic tissue, increasing the risks of effects. In most of these procedures, only some points of the skin will be treated, when it would be sufficient to anesthetize these few points instead of anesthetizing the entire region. Only once the region has been previously anesthetized can the site then be cleaned with the removal of the anesthetic and the marking of the specific site to be treated. With a view to reducing the time for these procedures, minimize the unnecessary use of large quantities of anesthetics, consequently, and increasing the safety of the procedure, the present invention combined these two steps: marking+topical anesthesia, enabling the creation of products that signal the sites that will be treated and at the same time selectively anesthetize the necessary points, resulting in efficiency and safety for the patients.

### STATE OF THE ART

Dyes for marking various surfaces of biological/organic tissues are known in the art. In some embodiments, said dyes are applied with the use of pens for marking biological tissue, such as for example, those made by the company Viscot, with dyes in the colors green, red and white, or also by other manufacturers such as Medline, Medchoice, etc.

Prior anesthesia in general is required before most minimally invasive or invasive procedures. The procedures for marking biological/organic tissue, such as skin, mucosae, among others, are currently carried out after the application of topical anesthetics throughout the area, which are subsequently removed, whereupon the biological tissue is marked and the being initiated. However, the possibility of manufacturing a dye for specifically marking organic or biological tissues containing anesthetics in its composition would save on the amount of anesthetic to be used and would decrease the area exposed to the topical anesthetic, minimizing side effects, saving time for the professional and, at the same time providing more comfort and safety for the individuals who subsequently undergo said procedures.

Topical anesthetics, broadly used in medical, surgical and cosmetic procedures present an exclusive analgesic effect, without any other function, and present various uses and forms of application, the most common in these cases being the application in creams or ointments containing one or more topical anesthetics in the formulation, and are applied throughout the area where the procedure will be carried out, with due regard for the amount limit that can be used with safety.

In some minimally invasive cosmetic procedures, it is common for various areas of a patient to require prior anesthesia before a procedure, such as, for example, multiple micropunctures. After the physical examination of the area and planning of the procedure to be adopted, a topical anesthetic is normally applied commonly known in the art over a broad area of the biological tissue that will be treated, such as the entire face. Often times, topical anesthetics applied throughout the face of a patient may trigger the excessive absorption of anesthetics, leading to dangerous toxicity to the patient. Further, there are procedures that are carried out simultaneously on the face and on other areas of the body, a fact that would require the use of topical anesthetics in even larger areas than the face, today limiting serious risks to the health of the individual. After anesthetizing the biological tissue, the professional qualified to perform the procedure marks with a regular marking pen the regions to be treated.

Patent document US2008131527—TOPICAL ANALGESIC COMPOSITION presents a topical analgesic composition for use on a significant open wound, said composition comprising at least one local anesthetic agent and a carrier to form a long-lasting barrier over the open wound, and to promote and prolong the contact of the anesthetic agent with the wound, wherein the composition provides an analgesic effect beyond that which is attributed to the anesthetic agent, while the barrier covers the wound. The composition can be applied as a spray in gel, emulsion, powder, solution, cream, suspension or foam, so as to disturb the injury as little as possible. Besides these characteristics, it may contain a marker to identify the application of the product on the biological tissue, and the marker may be a dye/pigment. Said document does not disclose a composition to be applied on integral tissues and does not have the function of anesthetizing only small points that will be treated, since the dye used as marker is mixed to the solution so as to spread jointly with the anesthetic, having the sole function of identifying the diffusion reach thereof on the biological tissue, and not intentionally mark the tissue before carrying out subsequent surgical or cosmetic procedures. In this case the objective is an anesthesia in extensive wound region (non-integral biological tissue), with long-lasting time, quite different to the current description where analgesia is sought with quick-start and short-lasting action, as there is no need to last more than the time to carry out the procedure, the marking limited exclusively to the points necessary. All this results in a safer, more effective local anesthesia, as the amount and the extent of the biological tissue to receive said anesthetics are much less.

Patent document KR20020026402—COMPOSITION FOR TOPICAL APPLICATION CONTAINING LOCAL ANESTHESIA AS ACTIVE INGREDIENT AND PYRROLIDINE DERIVATIVE AS SKIN PERMEATION ENHANCER presents a composition for topical application, whose composition contains a local anesthetic as an active ingredient and a pyrrolidine derivative as skin permeation enhancer to improve the rate of skin permeation. The composition for topical application contains: 0.2-20% by weight of local anesthetic, as active ingredient and 5-30% by weight of a pyrrolidine derivative, as a skin permeation enhancer. It also contains 5-50% by weight of solvent and 0.5-25% by weight of thickener. Additionally, it contains pharmaceutically acceptable additives, such as moisturizing agents, pigments, preservatives, stabilizing agents, buffer, pH regulating agents and the like. Said document does not present a pigment in its composition responsible for prior marking of a patient's biological tissue, not serving as anesthetic skin marker, as the one described.

Patent document US2008085245—TOPICAL ANESTHETIC COMPOSITION presents a method for providing anesthesia to an individual with a wound, such as a laceration, a surgical incision, an ulcer, an abrasion or a burn. The method comprises topically applying to the wound a composition comprising: at least one local anesthetic agent; a hydrophillic or hydroalcoholic gelling agent; an antiseptic agent; a vasoconstrictor; and a marker detectable as a food dye, in that when the composition is applied topically to the wound of the individual, the presence of the anesthetic agent in the subject is indicated by the detectable marker. The method was developed chiefly to anesthetize open wounds in animals. Said method is not applicable for effective marking of sites for subsequent performance of medical, surgical or cosmetic procedures, since the function of the dye adopted is to indicate the presence of the anesthetic agent, and not to remain in the region marked on the surface of the tissue to delimit the anesthetized site; additionally, said method has application in open wounds, not solving the technical problem of guaranteeing the absorption of the anesthetic and the non-absorption of the pigment in integral organic tissues, for example.

Patent document WO2007022090—TOPICAL DELIVERY WITH A CARRIER FLUID presents formulations of aerosol in spray capable of providing a high concentration of materials containing active agent and /or excipient. The active ingredient may be any pharmaceutically active agent, but is preferably an antibiotic, an anti-histamine, an anesthetic, an anti-inflammatory and/or an adstringent. The formulation may also contain one or more pharmacologically acceptable excipients, such as antioxidants, stabilizers, perfumes, dyes, viscosifiers, emulsifiers, surfactants and combinations thereof. Said formulations do not permit a fine marking of regions of organic tissue to be subjected to invasive and minimally invasive medical, surgical or cosmetic treatments on biological tissue, and nor does it guarantee effective action of the anesthetic through the tissue where it is applied.

The North American patent application document US 2018/0000757 presents an anesthetic composition containing a mixture of lidocaine and tetracaine for manufacturing a solid local anesthetic further comprising polyvinyl alcohol, water and an emulsifying agent. One of the drawbacks of this invention is that said composition should be cooled. The present invention differs from this patent application since it provides an anesthetic composition and dye that can be delivered to the organic tissue which comprises a composition different to this prior art and also contains a pigment for marking the site to be treated, causing a localized analgesia and in a safe manner.

The anesthetic made by the company Galderma, known as Pliaglis, presents a composition of 7% of lidocaine and 7% of tetracaine for topical use on organic tissues of patients in need of analgesia. It is stated in the instructions of this formulation that said composition needs cooling. Nor is the possibility of coloring said cream disclosed in any patent document related to this invention. Therefore, the present invention is also quite different from this product, since the present composition of the invention can be stored at ambient temperature and contains pigments. Therefore, the compositions disclosed in this document serve to anesthetize and mark the site to be treated, optimizing the amount of anesthetic to be applied, whereby enhancing the safety of the procedure.

The international publication WO 2019057822 discloses in its formulation an anesthetic dye for tattoos, comprising a composition of 6 to 20% of anesthetics, preferably lidocaine and benzocaine. These anesthetics jointly with the pigments are applied by needles, the objective being to anesthetize the act of tattooing. Since the anesthetic is placed inside the skin jointly with the pigment used to tattoo the skin, the objective of the pigment in this case is really to tattoo the skin definitively, and not mark the site that will be anesthetized and subsequently treated by way of invasive or minimally invasive medical, surgical or cosmetic procedures. Therefore, this is a procedure where the anesthetic is placed inside the skin by way of needles, which increases the risk of serious adverse effects such as intoxication and does not bring precision to the site to be anesthetized.

Countless pharmacological factors determine the penetration and the efficacy of a pharmacologically active agent on the organic tissue, including liposolubility, the protein bonding capacity, the pKa and local vasodilation. Lipophilic molecules quickly spread across the epidermis and into the nerve endings. Once in the dermis, the molecules with high capacity to bond to proteins are more stable and determine more prolonged analgesia. In the current state of the art, there are documents that present topical anesthetics which have some form of dye/pigmentation for identifying the application thereof in organic tissue to solve the pain resulting from skin injury. However, dyes for identifying the presence of anesthetics in integral organic tissue cannot be effectively applied for the solution of the technical problem of marking regions to be subjected to invasive or minimally invasive medical, surgical or cosmetic procedures on biological tissue, insofar as the trajectory of the dyes, according to the state of the art, follows the trajectory of diffusion on the tissue of the pharmacologically active compounds applied, hampering a precise marking and causing unnecessary absorption of the dye through the organism of the patient. There is no guarantee that the dyes described in the state of the art allow a precise marking of the desired region, that do not tattoo the tissue, or that run-off, excessive absorption and consequent side effects do not occur, as well as wastage of the anesthetic during the marking of the biological tissue. Therefore, it is understood that no dye has yet been provided for marking organic tissue for invasive or minimally invasive medical, surgical or cosmetic procedures on biological/organic tissue that permits precise and stable marking, with fast but low absorption of the topical anesthetics, that is easily removable from the region, and, at the same time, performs fast and effective local anesthesia, with minimal absorption of the anesthetic by the tissue.

The dye for marking organic tissue with an anesthetic, disclosed in this specification, has a differentiated formulation responsible for guaranteeing the unexpected technical effects in light of the state of the art. It is an objective of the invention to anesthetize and mark, quickly and effectively, only the region that will undergo the invasive or minimally invasive medical, surgical or cosmetic procedures on biological tissue, causing discomfort or pain to the patient, using the composition of the present invention comprising, besides pigments for marking the desired region, topical anesthetics that leave the area anesthetized for the procedure. Alternatively, said composition may comprise products that accelerate the action of the anesthetic and guarantee improved pain control.

Another objective of the invention is to eliminate the double step of preparing the biological tissue to be anesthetized for painful procedures. Today, it is known to persons skilled in the art to pass a topical anesthetic throughout the region to be treated, wait for the effect thereof, clean the skin and only then mark the points where the procedures will be carried out. The present invention provides a composition for marking biological/organic tissue, such as skin, mucosa and any other tissues of living beings, comprising a dye or pigment, jointly with at least one anesthetic, in order to make medical, surgical and cosmetic procedures safer, faster and more practical.

Therefore, the provision of a dye for marking organic tissue with an anesthetic simplifies the invasive or minimally invasive medical, surgical or cosmetic procedures on biological tissue causing pain to individuals. Previously, this procedure needed to be done in four steps (anesthesia—removal of excess anesthetic—marking the skin—puncture) and now can be done in just two steps (marking with anesthesia —puncture). The function of the dye is to pigment the organic tissue, marking the region where the procedure will occur and which, concomitantly, will be anesthetized. The function of the anesthetics is to anesthetize the organic tissue marked with the dye and which will therefore undergo the procedure. Other products may optionally be added to this composition to accelerate the absorption of anesthetics, resulting in an anesthetic effect in a shorter time than that known in the art.

In an additional embodiment, a method is provided for applying said anesthetic dye, which confers increased certainty and accuracy that the point marked for the procedure is anesthetized. Said method of specifically marking sites where the procedure will be carried out prevents waste of the anesthetic and increases safety, chiefly when various regions are treated. Therefore, without this technique, the individual would be exposed to a large amount of anesthetic which could cause intoxication or even death. Said method enables the subsequent procedure to be painless because it precisely limits the points to be injected and prevents side effects resulting from the anesthesia of large areas of biological tissue.

### DETAILED DESCRIPTION OF THE INVENTION

In a first embodiment, the present invention proposes a dye composition for marking organic tissue that results in safe localized analgesia, comprising:
at least one pigment;
at least one fast action anesthetic;

and, optionally, at least one anesthesia accelerator.

In a given embodiment, the dye composition for marking organic tissue may be such that said at least one pigment remains on the outer surface of the organic tissue, marking it, while the topical anesthetic attains its target of action. According to one embodiment, said pigment can be solid, liquid, dissolved or not in a pharmacologically acceptable medium, etc. In one embodiment, the coloration of the pigment may be identified by specific light, for example, ultraviolet. Local anesthetics reduce the excitability of the nerve fibers, block the inflow of solid ions through the interaction with a link site inside the membrane of the channel of sodium ions and, therefore, prevent the formation of potential action mechanisms necessary for conducting the pain. The result is a blocking in the stimulus transmission of the nerve cells, causing local analgesia.

In one embodiment, a pigment is adopted that is not absorbed by the epidermis or other organic tissue where it is applied, as known by persons skilled in the art. Therefore, the topical anesthetic can cross the skin barrier and act on the nerve endings, while the pigment is not absorbed by the tissue, remaining on its surface for marking the site to be treated. This confers countless advantages, reducing the potential toxicological implications of absorbing a dye that may be absorbed jointly with the anesthetic, preventing the unintentional pigmentation of areas beyond the one where marking is desirable, and assuring improved visualization of the marked area to the health professional who will carry out the procedure on the biological tissue of an individual.

Particularly preferred local anesthetics are selected from the group consisting of lidocaine, mepivacaine, prilocaine, articaine, bupivacaine, dibucaine, ropivacaine, etidocaine, dyclonine, procaine, benzocaine, 2-chloroprocaine, oxybuprocaine, tetracaine, fomocaine, campocaine, Levobupivacaine, Oxyprocain, Hexylcain, Dibucaine, Piperocaine, Butamben, Butamben picrate, Dimethisoquin hydrochloride, Diperodon, Dyclonin, Ketamine, p-Butylaminobenzoesaure, Pramoxin and pharmacologically acceptable salts thereof, as well as mixtures thereof. Said anesthetics are divided into amide-based and ester-based anesthetics. The most common amide-based anesthetics include lidocaine, prilocaine, mepivacaine, etidocaine, bupivacaine, ropivacaine, levobupivacaine, or any combination thereof. The most used ester-based anesthetics are procaine, benzocaine and tetracaine, or a combination thereof.

In one embodiment, the composition of the invention comprises at least one amide-based anesthetic. In another embodiment, the anesthetic dye is provided with an ester-based anesthetic. In a third preferred embodiment, the anesthetic dye comprises a mixture of both bases, for example, one ester-based anesthetic and one amide-based anesthetic.

In one embodiment, said composition may comprise lidocaine in a concentration of 2 to 30% by weight as a first anesthetic and, optionally, tetracaine in a concentration of 3 to 30% by weight, or any combination thereof.

Preferably, a mixture of lidocaine and tetracaine is adopted. Preferably, the concentration of lidocaine is 10 to 20% by weight. More preferably, the concentration of lidocaine is 15% by weight. Preferably, concentration of tetracaine is 5 to 10% by weight. More preferably, the concentration of tetracaine is 7% by weight. These concentrations may vary in accordance with the sensitivity of the individual and whether the type of procedure to be carried out is slightly or very painful.

Lidocaine and tetracaine are classic combinations of medicines to produce local analgesia in the intact skin, prior to invasive or minimally invasive medical, surgical or cosmetic procedures to the skin. Lidocaine acts as anesthetic during the medical procedure. Other anesthetics can be used in the composition without prejudice, and the combination thereof is possible when it provably offers no risks to the patient.

In one embodiment, said at least one pigment is selected from: titanium oxide, zinc oxide, iron oxide, magnesium oxide, manganese oxide, copper EDTA chelate, pyrophyllite, acid red, magnesium silicate, guaiazulene, bromocresol green, bromothymol blue, aluminum stearate, zinc stearate, calcium stearate, anthocyanins, anthocyanins, caramel, lactoflavin, Hansa yellow pigment, permanent red pigment, permanent rubine pigment, BON red pigment, alizarin red pigment, perinone orange, ultramarine blue, ultramarine pink, ultramarine violet, manganese violet, Prussian blue, carbon black, amaranth lacquer, erythrosine lacquer, carmine lacquer, insoluble barium lacquers, strontium and zirconium, or any combination thereof.

In a preferred embodiment, said pigment is titanium oxide. Preferably, said pigment is provided in said composition in the form of a serum containing titanium oxide pigment. Preferably, said at least one pigment is a micronized pigment. Preferably, said at least one pigment is in a concentration between 0.5% and 20% by weight, more preferably between 1% and 10% by weight. Said pigment has the advantage of being hypoallergenic.

To enable improved efficiency in applying the product, it is important that it has a suitable viscosity, thus assuring that the product be viscous enough not to run off along the site applied (organic tissue) which could generate wastage and analgesia of undesirable regions, or even smudging the marking of the site and not correctly anesthetizing the desired site. Further, at the same time, said product cannot be too viscous as this would adversely affects its application on the organic tissue, and might even disturb the homogeneity of applying anesthesia on the tissue.

Therefore, the following measures may be adopted to achieve satisfactory viscosity characteristics. In one embodiment, a pigment can be adopted that, in dispersion, contributes to alter the viscosity of the product up to the desired levels. For example, a micronized inorganic pigment can be used including, for example, micronized iron oxides, micronized chrome oxides, or micronized titanium dioxides.

In a preferred embodiment, the compositions of the present invention are formulated to be delivered to the organic tissue through a device, such as a pen. In this case, if the pigment is not capable of conferring the desired viscosity to the anesthetic product, a gelling agent may optionally be incorporated to the mixture, in the quantity necessary to guarantee the suitable levels of viscosity, and provided that it does not diminished the desired analgesic effect. In a general manner, the gelling agent used can be, for example, selected from a group consisting of sodium polyacrylate, anionic polyelectrolytes, resins, clays, bentonite, carboxymethylcellulose, silicone resins, guar gum, xanthan gum, carob gum, acacia gum, or any combination thereof. Preferably, the viscosity of the composition is from 800 to 25 000 mPa·s, preferably between 800 and 10 000 mPa·s, more preferably between 800 and 6000 mPa·s. The concentrations of the gelling agent used may vary between 0.5% and 15% by weight, preferably between 0.6% and 6% by weight.

In one embodiment, it may be desirable for the fluid to have a thixotropic behavior, so that it can be stored in flasks or tubes, presenting a liquid behavior during its application. Accordingly, it may optionally be possible to provide for the addition of talcum, fumed silica or precipitated silica, or any combination thereof, in concentrations of 0.5% to 6% by weight. Other additives can be used to attain this objective, such as fats and vegetable oils.

In an additional embodiment, said at least one pigment is a visible pigment only under application of special light. In one embodiment, this special light may be called Wood light, which is an ultraviolet light produced jointly with a small amount of visible light. When the violet light acts on phosphorescent compounds, such as the mineral calcite or zinc sulfide, it is absorbed. The materials

In a preferred embodiment, the compositions of the present invention are formulated to be delivered to the organic tissue through a device, such as a pen. In this case, if the pigment is not capable of conferring the desired viscosity to the anesthetic product, a gelling agent may optionally be incorporated to the mixture in the amount necessary to guarantee suitable levels of viscosity, and provided that it does not diminish the desired analgesic effect. In a general manner, the gelling agent used can be, for example, selected from a group consisting of sodium polyacrylate, anionic polyelectrolytes, resins, clays, bentonite, carboxymethylcellulose, silicone resins, guar gum, xanthan gum, carob gum, acacia gum, or any combination thereof. Preferably, the viscosity of the composition is from 800 to 25 000 MPa·s⁻¹, preferably between 800 and 10 000 MPa·s⁻¹, more preferably between 800 and 6000 MPa·s⁻¹. The concentrations of the gelling agent used may vary between 0.5% and 15% by weight, preferably between 0.6% and 6% by weight.

In one embodiment, it may be desirable for the fluid to have a thixotropic behavior, so that it can be stored in flasks or tubes, presenting a liquid behavior during its application. Accordingly, it may optionally be possible to provide for the addition of talcum, fumed silica or precipitated silica, or any combination thereof, in concentrations of 0.5% to 6% by weight. Other additives may be used to attain this objective, such as fats or vegetable oils.

In an additional embodiment, said at least one pigment is a visible pigment only under application of special light. In one embodiment, this special light may be called Wood light, which is an ultraviolet light produced jointly with a small quantity of visible light. When the violet light acts on phosphorescent compounds, such as the mineral calcite or zinc sulfide, it is absorbed. The phosphorescent materials slowly emit part of the energy absorbed in the form of visible light, displaying a weak sheen on the marked site. This embodiment is applied in procedures in which the pigmentation of the region of the organic tissue might interfere with the procedure itself. By applying a phosphorescent pigment, for example, only by means of special light, such as black light, is it possible to visualize the marking, it being possible to carry out solely the processes that need visualization of the marking dye under the application of this special light, the remainder of the procedure being carried out without the application thereof, where the dye then becomes invisible, preventing any disturbances to the performance of the remainder of the procedure.

In one embodiment, said at least a pigment is volatile, remaining for a short period of time on the surface of the organic tissue to undergo the procedure of interest. In this case, the pigment naturally evaporates without leaving significant residues. In one embodiment, the pigment is volatile only when its temperature increases after receiving a certain thermal stimulus. In certain embodiments, the pigment may be volatile when subject to temperatures over 40° C.

In one embodiment, the composition may further comprise a moisturizer selected propylene glycol, glycerine, sorbitol and butylglycol, or a mixture thereof. The presence of a moistener may collaborate to facilitate the application of the product in the tissue and prevent the formation of crusts.

In one embodiment, the composition may further comprise a preservative selected from: imidazolidinyl urea; diazolidinyl urea; benzic acid; sodium benzoate; sorbic acid; potassium sorbate; propylparabene; methylparabene; ethylparabene; butylparabene.

In one embodiment, the composition may further comprise a vasoconstrictor selected from apraclonidine, brimonidine, clonidine, desglymidodrine, dexmedetomidine, dopamine, ephedrine, epinephrine, epinine (N-methyl-dopamine), ethylnorepinephrine, phenylephrine, phenylpropanolamine, guanabenz, guanfacine, I-dobutamine, levarterenol, lofexidine, mephentermine, metaraminol, methylphenidate, metoxamine, midodrine, mytodrine, mivazerol, moxonidine, naphazoline, norepinephrine, norphenylephrine, oxymetazoline, pemoline propylexedrine, propylhexedrine, tetryzoline, tizanidine, xylometazoline, α-methyldopa, α-methylnorepinephrine, (4,5-dihydro-1Himidazole-2-yl)-(8-methyl-quinoxaline-6-yl)-amine, (4,5-dihydro-1 H-imidazole-2-yl)-quinoxaline-5-yl-amine, (5-bromo-2-methoxy-quinoxaline-6-yl)-(4,5-dihydro-1 H-imidazole-2-yl)-amine, (5-bromo-3-methyl-quinoxaline-6-yl)-(4,5-dihydro-1 H-imidazole-2-yl)-amine, (8-bromo-quinoxaline-5-yl)-(4,5-dihydro-1 H-imidazole-2-yl)-amine, (8-bromoquinoxaline-6-yl)-(4,5-dihydro-1 H-imidazole-2-yl)-amine, or any combination thereof. The vasoconstrictor contributes to prevent unnecessary dissemination of the anesthetic to beyond the application region of interest.

The inclusion of an action accelerator agent may be an important part of the present invention to promote faster action of the anesthetic through the biological tissue. The anesthetic action accelerator can be selected from Transcutol® (compound of Ethoxydiglycol Diethylene Glycol Monoethyl ether) or products obtained from urea, azone, fatty acid esters, benzoate esters, n-methyl-2-pyrrolidone, dimethyl sulfoxide, menthol, cyclodextrins, oleic acid, phospholipids or any combination thereof. In a preferred embodiment, the concentration of the Transcutol® is between 5% and 91%, preferably between 10% and 68%, and more preferably in an amount of 15%. Therefore, the anesthetic may be absorbed by the tissue, while the pigment remains on the surface marking the area where the procedure will occur.

Preferably, a serum is adopted as carrier of the composition. In one embodiment, the composition may be in the form of liquid, lotion, gel, gel-cream, ointment, mousse, paste, oily or aqueous solution, powder or baton.

There are various forms in which said anesthetic dye may be provided to the organic tissue. For example, by brush, pen, ampoule, application rod, etc. Preferably, said dye composition is applied with a pen or stick manual, assuring greater accuracy in marking the region or the points of interest on the organic tissue.

In a preferred embodiment, said composition is produced such that it may be delivered continuously to the surface of the organic tissue by a pen, that is, its fluidity characteristics should be taken into consideration so that the composition is not too fluid to drain through the pen, nor too viscous that it cannot flow to the tip of said pen. In an optional embodiment, the pigment and the anesthetic may be mixed in just one phase, so that these may be applied as a single product visibly, but that the pigment remains on the surface of the biological tissue marking the site and the anesthetic penetrates in its site of action.

Preferably, said organic tissue is a skin surface, preferably of a human being. However, the composition may be used in any procedure that causes pain or discomfort, and where topical anesthesia is important to be achieved.

In another object of the present invention, a method of applying dye for marking organic tissue is provided for, comprising the steps of:
defining an organic tissue in need of marking for subsequent invasive or minimally invasive medical, surgical or cosmetic procedure;
providing a device containing an anesthetic composition comprising at least one anesthetic, at least one pigment, and, optionally, an anesthetic action accelerator, as described previously;
placing the tip of the device in contact with the biological tissue intended for marking and anesthetizing;
applying the dye comprising anesthetic on the surface of the biological/organic tissue of an individual who will undergo a procedure that causes pain or discomfort;
waiting around 5 to 30 minutes for the anesthetic to act upon the organic tissue, while the medical, surgical or cosmetic material is being prepared for the start of the procedure; and,
carrying out the procedure that causes pain or discomfort to the individual exactly in the marked area.

Preferably, in carrying out this method, the composition described in this document is used.

In one embodiment, this method may also include a step of removing said dye that is not absorbed by the organic tissue, such as a lipophillic dye. In another embodiment, the dye is volatile and evaporates when subject to a certain temperature, for example, over 40° C. In another example, the dye is detectable by a special light, such as an ultraviolet light. As a matter of description, it should be understood that other embodiments of dyes and dye carriers can be added to the composition of the present invention, without straying from the scope thereof.

A list of dyes that can be used, without the aim of limitation, includes titanium oxide, zinc oxide, iron oxide, magnesium oxide, manganese oxide, copper EDTA chelate, pyrophyllite, acid red, magnesium silicate, guaiazulene, bromocresol green, bromothymol blue, aluminum stearate, zinc stearate, calcium stearate, anthocyanins, anthocyanins, caramel, lactoflavin, Hansa yellow pigment, permanent red pigment, permanent rubine pigment, BON red pigment, alizarin red pigment, perinone orange, ultramarine blue, ultramarine pink, ultramarine violet, manganese violet, Prussian blue, carbon black, amaranth, erythrosine, carmine, insoluble barium lacquers, strontium and zirconium, gold, silver, iron hydroxide, bordeaux, ponceau 4R, AC red, patent blue, indigotine, indigo carmine, brilliant blue FCF, chlorophyll, chlorophyllin, cupric chlorophyll, sodium and potassium salts, caramel I-IV, aluminum, sunset yellow FCF, brilliant yellow, riboflavin, tartrazine, carminic acid, cochineal, synthetic and natural beta-carotene, annatto, bixin, norbixin, urucum, roku, paprika, capsorrubin, capsanthin, lycopene, beta-apo-8′-carotenal, beta acid methyl or ethyl ester, lutein, red beet, betamine, calcium carbonate, turmeric, or any combination thereof.

This method, contrary to that commonly adopted in the state of the art, dispenses with an individual step of applying the anesthetic, saving time and supplies and reducing waste and risks as only the necessary amount of the anesthetic is applied. After analyzing the individual, the innovative marking takes place only at the sites to be treated. Thereafter, while the necessary materials are prepared for the procedure, the anesthetic begins to act. When the material is ready and the time comes for performing the invasive or minimally invasive medical, surgical or cosmetic procedure on biological tissue, the patient already has the site anesthetized and the procedure takes place in a rapid, efficient and safe manner.

Therefore, the unexpected characteristics is foreseen of including anesthetic compositions in an ideal mixture jointly with a pigment or dye, to produce a dye composition for marking organic tissue, attaining an ideal viscosity so that it can be delivered by a single device, for example, a marker pen known in the state of the art, thus enabling two steps common in cosmetic, surgical or medical procedures (anesthesia and marking) to be combined into just one.

The following examples describe an embodiment of the formulation carried out according to the present invention.

### Example 1

A composition was prepared using the serum as carrier, lidocaine in a final concentration of 15% by weight, tetracaine in a concentration of 23% by weight, and micronized titanium oxide in a concentration of 10% by weight. Initially, the two anesthetics were mixed together, then mixed with the other ingredients whilst stirring. The serum provided firmness in the biological tissue during the procedure. The pigment effectively marked the contact area with the anesthetic active principles, acting as reference for the health professional, without dripping and anesthetizing the site to be treated, causing significant comfort to the individual undergoing the procedure.

### Example 2

A composition was prepared using the serum as carrier, the action accelerator agent Transcutol® 15%, lidocaine in final concentration of 5% by weight, tetracaine in a concentration of 3% by weight, and micronized titanium oxide in a concentration of 1% by weight. Initially, the two anesthetics were mixed together, then mixed with the other ingredients whilst stirring. The anesthetics were absorbed satisfactorily. The pigment effectively marked the contact area with the anesthetic active principles, acting as reference for the health professional, without dripping and anesthetizing the site to be treated, preventing pain and discomfort to the individual undergoing the procedure.

### Example 3

A composition was prepared using the serum as carrier, as action accelerator Transcutol® 15%, lidocaine in a final concentration of 15% by weight, tetracaine in a concentration of 7% by weight, and micronized titanium oxide in a concentration of 5.5% by weight. Initially, the two anesthetics were mixed together, then mixed with the other ingredients whilst stirring. The anesthetics were absorbed satisfactorily. The pigment effectively marked the contact area with the anesthetic active principles, acting as reference for the health professional, without dripping and anesthetizing the site to be treated, causing significant comfort to the individual undergoing the procedure.

### Constructive Embodiments of the Invention

One constructive embodiment presents a dye composition for marking organic tissue comprising a pigment mixed with an anesthetic, presenting a concentration of 5-30% by weight of a first amide-based anesthetic compound selected from the group consisting of lidocaine, prilocaine, mepivacaine, etidocaine, bupivacaine, ropivacaine, levobupivacaine; 3-25% by weight of a second compound ester-based anesthetic selected from the group consisting of procaine and tetracaine; and, a pharmacologically acceptable serum comprising at least one pigment.

In a preferred dye composition for marking organic tissue, said dye may additionally comprise at least one anesthetic action accelerator selected from the group consisting of compounds obtained from ethoxydiglycol diethylene glycol monoethyl ether, urea, azone, fatty acid esters, benzoate esters, n-methyl-2-pyrrolidone, dimethyl sulfoxide, menthol, cyclodextrins, oleic acid, phospholipids or any combination thereof. More specifically, said at least one anesthetic action accelerator is a ethoxydiglycol diethylene glycol monoethyl ether-based compound.

In preferred embodiments, said at least one pigment does not penetrate into the organic tissue and remains temporarily on the outer surface of the organic tissue while the anesthetic compound is absorbed by the tissue which is then anesthetized.

In another preferred embodiment the first anesthetic compound is lidocaine in a concentration of 10 to 20% by weight and the second anesthetic compound is tetracaine at a concentration of 5 to 15% by weight.

In a general manner, with said composition, any one of the pigments selected from the following group can be used, namely titanium oxide, zinc oxide, iron oxide, magnesium oxide, manganese oxide, copper EDTA chelate, pyrophyllite, acid red, magnesium silicate, guaiazulene, bromocresol green, bromothymol blue, aluminum stearate, zinc stearate, calcium stearate, anthocyanins, anthocyanins, caramel, lactoflavin, Hansa yellow pigment, permanent red pigment, permanent rubine pigment, BON red pigment, alizarin red pigment, perinone orange, ultramarine blue, ultramarine pink, ultramarine violet, manganese violet, Prussian blue, carbon black, amaranth, erythrosine, carmine, insoluble barium lacquers, strontium and zirconium, gold, silver, iron hydroxide, bordeaux, ponceau 4R, AC red, patent blue, indigotine, indigo carmine, brilliant blue FCF, chlorophyll, chlorophyllin, cupric chlorophyll, sodium and potassium salts, caramel I-IV, aluminum, sunset yellow FCF, brilliant yellow, riboflavin, tartrazine, carminic acid, cochineal, synthetic and natural beta-carotene, annatto, bixin, norbixin, urucum, roku, paprika, capsorrubin, capsanthin, lycopene, beta-apo-8′-carotenal, beta acid methyl or ethyl ester, lutein, red beet, betamine, calcium carbonate, turmeric, or any combination thereof. In a preferred execution of said invention, titanium oxide was used in a concentration between 0.5% and 20% by weight, more preferably, 10%. The pigments can generally be chosen from among liquids and powders, in which pigments that are not absorbed by organic technique and are visible to the naked eye, by visible light or by ultraviolet light are deemed preferable. Micronized pigments in powder are designated to this use, such as micronized pigments.

In alternative embodiments, said anesthetic dye may further comprise a gelling agent selected from sodium polyacrylate, anionic polyelectrolytes, resins, clays, bentonite, carboxymethylcellulose, silicone resins, guar gum, xanthan gum, carob gum, acacia gum, talcum, fumed silica or precipitated silica or any combination thereof, in a concentration of 0.5% to 6% by weight. Compositions can also be added to said moisturizers selected from propylene glycol, glycerine, sorbitol, butylcerol or a mixture thereof. To improve the shelf-life of the product, preservatives can be added to the composition in one embodiment, selected from: imidazolidinyl urea; diazolidinyl urea; benzic acid; sodium benzoate; sorbic acid; potassium sorbate; propylparabene; methylparabene; ethylparabene; butylparabene.

In alternative embodiments of the present invention, vasoconstrictors may also be added, selected from the group consisting of apraclonidine, brimonidine, clonidine, desglymidodrine, dexmedetomidine, dopamine, ephedrine, epinephrine, epinine (N-methyl-dopamine), ethylnorepinephrine, phenylephrine, phenylpropanolamine, guanabenz, guanfacine, 1-dobutamine, levarterenol, lofexidine, mephentermine, metaraminol, methylphenidate, metoxamine, midodrine, mytodrine, mivazerol, moxonidine, naphazoline, norepinephrine, norphenylephrine, oxymetazoline, pemoline propylexedrine, propylhexedrine, tetryzoline, tizanidine, xylometazoline, α-methyldopa, α-methylnorepinephrine, (4,5-dihydro-IH-imidazole-2-yl)-(8-methyl-quinoxaline-6-yl)-amine, (4,5-dihydro-IH-imidazole-2-yl)-quinoxaline-5-yl-amine, (5-bromo-2-methoxy-quinoxaline-6-yl)-(4,5-dihydro-IH-imidazole-2-yl)-amine, (5-bromo-3-methyl-quinoxaline-6-yl)-(4,5-dihydro-IH-imidazole-2-yl)-amine, (8-bromo-quinoxaline-5-yl)-(4,5-dihydro-IH-imidazole-2-yl)-amine, (8-bromoquinoxaline-6-yl)-(4,5-dihydro-IH-imidazole-2-yl)-amine, or any combination thereof.

For application of the dye on an organic tissue surface, devices are foreseen that act as a marker pen, in which an amount of dye is stored inside the device and when it comes into contact with an organic surface in need of anesthesia, such as skin tissue or mucosa, said device can release, homogeneously, the composition described in the present document. Other types of devices that allow the continuous marking of an organic tissue are also provided within the scope of the present invention, such as a stick, brush or tube for marking.

The present invention further has a method for applying the dye composition for marking organic tissue comprising the steps of:

a) selecting an organic tissue of a patient who will undergo a procedure with the need of local anesthesia;

b) applying the dye composition for marking as defined in claim 1, to said organic tissue;

c) waiting between 5 and 30 minutes for the tissue to be treated to be anesthetized;

d) carrying out an invasive or minimally invasive procedure in the marked area.

Although the examples refer to micronized pigments, other compositions could be adopted, including gelling agents, for example, provided that they guarantee the rheological properties previously mentioned in this document.

It is important to emphasize that the description set out does not have the weight of limiting the forms of execution of the inventive concept now proposed, but rather to illustrate and make the conceptual innovations disclosed in this solution understandable. Therefore, the descriptions should be interpreted illustratively and not in a limitative manner, as there may be other equivalent or analogous ways of implementing the inventive concept now disclosed and that do not stray from the scope of protection delineated in the solution proposed.

The present specification addressed a dye composition for marking integral organic tissue, comprising at least one anesthetic and at least one pigment, endowed with novelty, inventive activity, full disclosure, industrial application and, consequently, satisfying all the essential requirements for the grant of the privilege claimed.

What is claimed is:

1. A dye composition for temporary marking of organic tissue comprising;
at least one pigment mixed with two anesthetics, further comprising;
5-30% by weight of a first anesthetic amide-based compound selected from the group consisting of lidocaine, prilocaine, mepivacaine, etidocaine, bupivacaine, ropivacaine, and levobupivacaine;
3-25% by weight of a second anesthetic ester-based compound selected from the group consisting of procaine and tetracaine;
a pharmacologically acceptable carrier comprising water and glycerine, said carrier having a density between 0.95 and 1.20 g/ml and pH between 4.5 and 6.5; and
at least one insoluble pigment.

2. The composition of claim 1, further comprising at least one anesthetic action accelerator selected from the group consisting of: ethoxydiglycol diethyleneglycol monoethyl ether, urea, azone, fatty acid esters, benzoate esters, n-methyl-2-pyrrolidone, dimethyl sulfoxide, menthol, cyclodextrins, oleic acid, phospholipids or any combination thereof.

3. The composition of claim 2, wherein said at least one anesthetic action accelerator is an ethoxydiglycol diethyleneglycol monoethyl ether-based compound.

4. The composition of claim 1, wherein said at least one pigment does not penetrate the outer surface of the organic tissue and the anesthetic compound is absorbed by the organic tissue.

5. The composition of claim 1, wherein said first anesthetic compound is lidocaine in a concentration of 10 to 20% by weight.

6. The composition of claim 1, wherein said second anesthetic compound is tetracaine at a concentration of 5 to 15% by weight.

7. The composition of claim 1, wherein said at least one pigment is selected from the group consisting of: titanium dioxide, zinc oxide, iron oxide, magnesium oxide, manganese oxide, copper EDTA chelate, pyrophyllite, acid red, magnesium silicate, guaiazulene, bromocresol green, aluminum stearate, zinc stearate, calcium stearate, anthocyanins, lactoflavin, Hansa yellow pigment, permanent red pigment, permanent rubine pigment, BON red pigment, alizarin red pigment, perinone orange, ultramarine blue, ultramarine pink, ultramarine violet, manganese violet, Prussian blue, carbon black, amaranth, erythrosine, carmine, insoluble barium lacquers, strontium and zirconium, gold, silver, iron hydroxide, bordeaux, ponceau 4R, AC red, patent blue, indigotine, indigo carmine, brilliant blue FCF, chlorophyll, chlorophyllin, cupric chlorophyll, sodium and potassium salts, aluminum, sunset yellow FCF, brilliant yellow, riboflavin, tartrazine, carminic acid, cochineal, synthetic and natural beta-carotene, annatto, bixin, norbixin, urucum, roku, paprika, capsorrubin, capsanthin, lycopene, beta-apo-8′-carotenal, beta acid methyl or ethyl ester, lutein, red beet, betamine, calcium carbonate, turmeric, or any combination thereof.

8. The composition of claim 7, wherein said at least one pigment is titanium dioxide.

9. The composition of claim 1, wherein said at least one pigment is a micronized pigment.

10. The composition of claim 1, wherein said at least one pigment is in a concentration between 0.5% and 20% by weight.

11. The composition of claim 1, wherein said at least one pigment is a pigment visible only under application of special light, wherein said special light is visible or ultraviolet light.

12. The composition of claim 1, further comprising a gelling agent selected from the group consisting of: sodium polyacrylate, anionic polyelectrolytes, resins, clays, bentonite, carboxymethylcellulose, silicone resins, guar gum, xanthan gum, carob gum, acacia gum, talcum, fumed silica or precipitated silica or any combination thereof, in a concentration from 0.5% to 6% by weight.

13. The composition of claim 1, wherein said composition is a thixotropic composition.

14. The composition of claim 1, further comprising a moistener selected from the group consisting of carnauba wax, candelilla wax, ozokerite wax, propyleneglycol, and glycerine, or any combination thereof.

15. The composition of claim 1, further comprising a preservative selected from the group consisting of: imidazolidinyl urea, diazolidinyl urea, benzoic acid, sodium benzoate, sorbic acid, potassium sorbate, propylparabene, methylparabene, ethylparabene, and butylparabene.

16. The composition of claim 1, further comprising a vasoconstrictor selected from the group consisting of: apraclonidine, brimonidine, clonidine, desglymidodrine, dexmedetomidine, dopamine, ephedrine, epinephrine, epinine (N-methyl-dopamine), ethyl norepinephrine, phenylephrine, phenylpropanolamine, guanabenze, guanfacine, 1-dobutamine, levarterenol, lofexidine, mephentermine, metaraminol, methylphenidate, methoxamine, midodrine, mytodrine, mivazerole, moxonidine, naphazoline, norepinephrine, norphenylephrine, oxymetazoline, pemoline propylhexedrine, propylhexedrine, tetryzoline, tizanidine, xylometazoline, α-methyldope, α-methyl norepinephrine, (4,5-dihydro-1H-imidazole-2-yl)-(8-methyl-quinoxaline-6-yl)-amine, (4,5-dihydro-1H-imidazole-2-yl)-quinoxalin-5-yl-amine, (5-bromo-2-methoxy-quinoxalin-6-yl)-(4,5-dihydro-1 H-imidazole-2-yl)-amine, (5-bromo-3-methyl-quinoxaline-6-yl)-(4,5-dihydro-1H-imidazole-2-yl)-amine, (8-bromo-quinoxalin-5-yl)-(4,5-dihydro-1H-imidazole-2-yl)-amine, (8-bromoquinoxaline-6-yl)-20(4,5-dihydro-1H-imidazole-2-yl)-amine, or any combination thereof.

17. The composition of claim 1, wherein said composition comprises a serum, liquid, lotion, gel, gel-cream, oily solution or aqueous solution.

18. The composition of claim 1, wherein said composition presents a viscosity of 800 to 25,000 mPa·s, preferably between 800 and 10,000 mPa·s, more preferably between 800 and 6000 mPa·s.

19. The composition of claim 1, wherein said organic tissue is a skin surface.

20. A cosmetic method for applying dye for temporary marking of organic tissue comprising the steps of:
a) selecting an organic tissue of a patient who will undergo a procedure with the need of local anesthesia;
b) applying the composition of claim 1 to said organic tissue;
c) waiting between 5 and 30 minutes, for the anesthesia of the organic tissue to be treated;
d) performing a cosmetic procedure on the marked surface of the anesthetized organic tissue
